(19) United States
(12) Patent Application Publication
(10) Pub. No.: US 2026/0115420 A1
(43) Pub. Date: Apr. 30, 2026

(54) FLEX CIRCUIT FOR AN INTRACARDIAC ECHOCARDIOGRAPHY CATHETER DEVICE

(71) Applicant: yoR Labs, Inc., Beaverton, OR (US)

(72) Inventors: Dakota Pellegrino, Hillsboro, OR (US); Blake W. Little, Fox Island, WA (US); Ariel Garcia, Vista, CA (US); Jessica Myrtle Chapman, Beaverton, OR (US); Anshumali Roy, Portland, OR (US)

(21) Appl. No.: 19/032,673

(22) Filed: Jan. 21, 2025

Related U.S. Application Data
(60) Provisional application No. 63/712,264, filed on Oct. 25, 2024.

(51) Int. Cl. A61M 25/00; A61M 39/00

(52) U.S. Cl. CPC A61M 25/0054; A61M 39/00

(57) ABSTRACT

A flexible catheter can include a distal region having a transducer attachment including a plurality of signal ribbons extending from the transducer attachment. The plurality of signal ribbons can each include a plurality of traces electrically coupled to and extending along a length of the plurality of signal ribbons. The flexible catheter can also include a medial region having a shielding section and a plurality of vias positioned along the shielding. A flexible catheter may include a proximal region having a set of long signal ribbons and a second set of short signal ribbons adjacent to the set of long signal ribbons. The proximal region can also include a plurality of connector pads positioned on the set of long signal ribbons and the second set of short signal ribbons.

### INCORPORATION BY REFERENCE TO ANY PRIORITY APPLICATIONS

This application claims the benefit of U.S. Provisional Application No. 63/712,264, filed Oct. 25, 2024, which is incorporated by reference herein in its entirety. Any and all applications for which a foreign or domestic priority claim is identified in the Application Data Sheet as filed with the present application are hereby incorporated by reference under 37 CFR 1.57 for all purposes and for all that they contain.

### BACKGROUND

### Field of the Invention

The present disclosure relates to a flex circuit for an intra-cardiac echography (“ICE”) catheter.

### Description of the Related Art

Intra-cardiac echography (“ICE”) catheters are widely used for diagnosing or assessing injured or diseased heart structures, tissues, or vessels, to determine if further treatment is needed. For example, as an ICE catheter is placed in or near the heart, an ultrasonic transducer array disposed on the surface of the catheter can emit ultrasonic pulses and/or signals which images the heart. To ensure the ICE catheter can effectively navigate through portions of tissues and/or vessels, a flexible circuit or flexible interconnect having a transducer arrangement disposed on the surface of the flexible circuit can provide imaging of the affected tissues or structures of a heart.

### SUMMARY

Embodiments of devices and methods for a flex circuit for an ICE catheter are disclosed.

In some embodiments disclosed herein, a flexible circuit for insertion into a catheter device is disclosed. The flexible circuit can include a distal region. The distal region can further include a transducer attachment region (or simply “transducer attachment”) including a plurality of traces. Additionally, the distal region can include a plurality of signal ribbons extending from the transducer attachment. Each of the plurality of signal ribbons can include a set of the plurality of traces electrically coupled to and extending along a length of the plurality of signal ribbons. The distal region can also include a first shielding section positioned above the plurality of signal ribbons in an unfolded configuration. The flexible circuit can also include a medial region. The medial region can include a second shielding section adjacent to the first shielding section. Additionally, the medial region can include a plurality of vias positioned along the second shielding section. The flexible circuit can also include a proximal region. The proximal region can include a set of long signal ribbons. The proximal region can also include a second set of short signal ribbons adjacent to the set of long signal ribbons. Additionally, the proximal region can include a plurality of connector pads positioned on the set of long signal ribbons and the second set of short signal ribbons. The flexible circuit can include a plurality of layers. The plurality of layers can include a first layer of the plurality of layers which may have a crosshatching section. The crosshatching section can be operable to connect ground regions between the plurality of signal ribbons. The plurality of vias can connect the ground regions across the plurality of layers. Additionally, the plurality of vias can have ground regions between at least a set of the plurality of signal ribbons.

In some embodiments disclosed herein, the plurality of traces can be sixty-four traces. Additionally, each set of the plurality of traces can be sixteen traces. In some embodiments, the transducer attachment can include a ground bar where at least a portion of the plurality of traces are coupled to the ground bar. The plurality of signal ribbons can include four signal ribbons, wherein each of the four signal ribbons is electrically coupled to a set of the plurality of traces, each set of the plurality of traces including sixteen traces.

In some embodiments disclosed herein, the first shielding section can include two shielding panels. Additionally, the crosshatching section can extend from a distal end of the plurality of signal ribbons. In some embodiments, the first layer of the plurality of layers is a top ground layer. The top ground layer can be made of copper. The plurality of layers may include five layers. The five layers can include a first layer which can be a top ground layer, a second layer which can be a top cover layer, a third layer which can be a base polyimide layer, a fourth layer which can be a bottom ground layer, a fifth layer which can be a bottom cover layer.

In some embodiments disclosed herein, the medial region can be longer than the distal region and the proximal region. Additionally, the second shielding section can include three panels. The second shielding section can include a plurality of slots configured to permit the flexible circuit to bend. The plurality of slots can be approximately 10 millimeters in length or approximately 15 millimeters in length.

In some embodiments disclosed herein, the plurality of signal ribbons can be configured to roll into a folded configuration for assembly in the catheter device. Additionally, the plurality of vias can include first vias and second vias, where the first vias form a cluster of three vias and the second vias can form a cluster of five vias. The first vias and the second vias can be positioned along the second shielding section to reduce the distance between ground regions when the flexible circuit is in a folded configuration.

In some embodiments disclosed herein, the plurality of traces can extend from the transducer attachment, along an entire length of the plurality of signal ribbons, and terminate at the plurality of connector pads. The set of long signal ribbons and the second set of short signal ribbons can each include one or more cut tabs. The distal region can also include a two panel shield region. The crosshatching section can include gaps operable to provide flexibility along the length of the plurality of signal ribbons. Additionally, the plurality of signal ribbons can have a width of approximately 1.5 mm.

In some embodiments disclosed herein, the plurality of connector pads can include a third set of vias. The plurality of traces extending from the transducer attachment can terminate at the third set of vias. The first shielding section and the second shielding section can be configured to wrap around the plurality of signal ribbons. Additionally, the flexible circuit can include one or more sensors positioned on the medial region and the proximal region.

In some embodiments disclosed herein, a fishing hole can extend through the plurality of signal ribbons in the proximal region. The fishing hole can be configured to guide the flexible circuit inside the catheter device during assembly.

In another embodiment disclosed herein, a flexible circuit for insertion into a catheter device is disclosed. The flexible circuit can have a distal region. The distal region can have a transducer attachment including a plurality of traces. The distal region can have a plurality of signal ribbons extending from the transducer attachment. Each of the plurality of signal ribbons can include a set of the plurality of traces electrically coupled to and extending along a length of the plurality of signal ribbons. The first shielding section can be positioned above the plurality of signal ribbons in an unfolded configuration. The flexible circuit can also have a medial region. The medial region can include a second shielding section adjacent to the first shielding section. The medial region can also include a plurality of vias positioned along the second shielding section. The flexible circuit can include a proximal region including a plurality of connector pads positioned on the plurality of signal ribbons. The flexible circuit can include a plurality of layers. The first layer of the plurality of layers can include a crosshatching section. The crosshatching section may connect ground regions between the plurality of signal ribbons. The plurality of vias may be operable to connect the ground regions between at least a set of the plurality of signal ribbons.

In some embodiments disclosed herein, a method of assembly of a flexible circuit for insertion in a catheter device is disclosed. The method can include obtaining a flexible circuit in an unfolded state. The flexible circuit can have a distal region including a transducer attachment including a plurality of traces. The distal region can have a plurality of signal ribbons extending from the transducer attachment. Each of the plurality of signal ribbons can include a set of the plurality of traces electrically coupled to and extending along a length of the plurality of signal ribbons. The distal region can also have a first shielding section. The flexible circuit can have a medial region which can have a second shielding section including a plurality of vias. The flexible circuit can have a proximal region including a plurality of connector pads. The method can include wrapping the flexible circuit from the unfolded state. Wrapping the flexible circuit can include stacking each signal ribbon of the plurality of signal ribbons. The first shielding section and the second shielding section can be positioned externally to the plurality of signal ribbons in a folded state. Additionally, the first shielding section and the second shielding section are configured to protect the plurality of signal ribbons.

### BRIEF DESCRIPTION OF THE DRAWINGS

FIG. 1 illustrates top view of a flexible circuit in an unfolded configuration for use in an intracardiac echocardiography (“ICE”) catheter device having a transducer attachment region at a distal end, a plurality of signal ribbons extending along the length of the flexible circuit, and a connector region at a proximal end.

FIGS. 2A-2E illustrate one or more layers of the flexible circuit of FIG. 1 which can include a bottom cover layer, a bottom ground (e.g., copper) layer, a base layer, a top ground (e.g., copper) layer, and a top cover layer.

FIG. 3A illustrates a bottom view of the flexible circuit of FIG. 1.

FIG. 3B illustrates a section view of a medial region of the flexible circuit of FIG. 3A.

FIG. 4A illustrates a top view of a distal region of the flexible circuit of FIG. 1.

FIGS. 4B-4C illustrate a bottom view of a distal region of the flexible circuit of FIG. 1.

FIG. 4D illustrates a top view of a layer of the flexible circuit in the distal region in FIG. 1 having a crosshatching region.

FIG. 4E illustrates a detailed section view of the layer of the flexible circuit in FIG. 4D.

FIG. 4F illustrates a top sectional view of a layer of the flexible circuit in FIG. 4D.

FIG. 5A illustrates a top view of a medial region of the flexible circuit of FIG. 1 having a shielding section.

FIGS. 5B-5C illustrate a layer of a medial region of the flexible circuit of FIG. 1 having a shielding section.

FIG. 5D illustrates a detailed view of the medial region of the flexible circuit of FIG. 1 having a shielding section.

FIG. 5E illustrates a layer of a medial region of the flexible circuit of FIG. 1 having a shielding section.

FIGS. 6A-6E illustrate a view of the proximal region of the flexible circuit of FIG. 1.

FIG. 7A illustrates a perspective top view of the flexible circuit of FIG. 1 in a folded configuration.

FIG. 7B illustrates a cross-sectional view of the flexible circuit in the folded configuration of FIG. 7A.

FIG. 7C illustrates a cross-sectional view of the flexible circuit in the folded configuration of FIG. 7A.

FIG. 8A illustrates a detailed top view of the distal region of a flexible circuit.

FIG. 8B illustrates a top sectional view of the distal region of the flexible circuit in FIG. 8A.

FIG. 8C illustrates a detailed top view of the distal region of the flexible circuit.

FIG. 8D illustrates a top sectional view of the distal region of the flexible circuit

FIG. 8E illustrates a top sectional view of the distal region of the flexible circuit.

FIG. 8F illustrates a detailed top view of the distal region of the flexible circuit.

### DETAILED DESCRIPTION

The following detailed description describes embodiments of flex circuits for use with an intra-cardiac echography (“ICE”) catheter and methods, some of which are illustrated in the figures. These embodiments are not intended to be limiting, and various modifications, variations, combinations, etc., of the features of these embodiments are possible and within the scope of this disclosure.

In some embodiments, the catheter devices and systems described herein can have an array of transducer attachments arranged on a surface of the catheter device. The transducers can emit ultrasonic energy to create an image of the tissue or vessel. Various embodiments of transducers can, using ultrasonic or doppler ultrasonic waves, determine an image of the tissue or vessel by transmitting ultrasound waves which impinge on vessel walls or tissue structures, receive ultrasound waves reflected from the vessel walls or tissue structure, generate a signal from the received waves, and send signals from the transducers to an imaging system of the catheter device.

In some embodiments, the catheter devices can have a flexible circuit or flexible member, which can be sized and shaped for insertion into a vessel or a tissue structure. In an example, a flexible circuit includes several layers including, but not limited to, base layers, conductive layers, and cover layers. The conductive layer can be copper or another conductive material. The flexible circuit can have a distal region and a proximal region. The flexible circuit can have a plurality of signal ribbons which extend from the distal region to the proximal region. The distal region can have an array of transducers (e.g., a transducer attachment). Furthermore, by having a flexible circuit instead of a rigid circuit, the imaging capabilities of the catheter device can improve, and the signals received and processed by the catheter device can better determine if there are any irregularities in the vessel or tissue structure (e.g., blood flow circulation problems, blood flow stoppage, etc.). The proximal region of the catheter device can have a cable connection portion and/or a series of connector pads, where the signals which are emitted and received by the transducer attachment can be received/sent to image the vessel where the transducer attachment is placed. In some embodiments, the signals can be relayed from the transducer attachment to the connector pads via a plurality of traces, where the plurality of traces can be a conductive material (e.g., copper). Furthermore, the plurality of traces can be arranged on the flexible circuit disposed between the connection pads and the transducer attachment. Advantageously, the size and arrangement of the plurality of traces can allow the signals emitted and received by the transducer array to have a shorter relay time between the transducer attachment and connection pads (e.g., due to shorter distances the signals travel) which can improve signal processing, signal processing time, and imaging).

In some embodiments, the flexible circuit and devices described herein can have a shield, or multiple shield regions. The shield or multiple shield regions can be used to partially protect or completely protect the flexible circuit. The shield or multiple shield regions can extend along the entire length of the signal ribbons. Additionally, when in a rolled configuration the shield can be located in a position to protect the cable connection portion. The shield or multiple shield regions can, in a rolled configuration, provide space for sensors to be attached to upper and lower regions of the flexible circuit.

Embodiments of a flexible circuit for use in a catheter (e.g., ICE catheter) are disclosed herein. The following is a list of certain components that are described and enumerated in this disclosure in reference to the above-listed figures. Other components, or aspects of these components, may not be included in the list but are disclosed in the figures and description. Accordingly, any aspect illustrated in the figures, whether or not called out separately herein, can form a portion of various embodiments and may provide basis for claim limitation relating to such aspects, with or without additional description. The enumerated components include:

| FIG. No. | Figure Description |
|---|---|
| 100 | Flexible Circuit |
| 101 | Bottom Cover Layer |
| 102 | Bottom Ground Layer |
| 103 | Base Polyimide Layer |
| 104 | Top Ground Layer |
| 105 | Top Cover Layer |
| 108 | Ground Bar |
| 109 | Hole |
| 110 | Distal End |
| 112 | Plurality of Traces |
| 113 | Upper Region |
| 114 | Transducer Attachment |
| 116 | Outer Edge |
| 118 | Tab |
| 122 | Cutout |
| 130 | Signal Ribbons |
| 130A | First Signal Ribbon |
| 130B | Second Signal Ribbon |
| 130C | Third Signal Ribbon |
| 130D | Fourth Signal Ribbon |
| 134 | Crosshatch |
| 138 | Gap |
| 140 | End of Coverlay |
| 150 | Middle Region |
| 152 | Via |
| 152A | Set of Three Vias |
| 152B | Set of Five Vias |
| 154 | Slot or Hole |
| 156 | Ground Shield |
| 156A | First Ground Shield |
| 156B | Second Ground Shield |
| 158 | Two Panel Shield |
| 160 | Three Panel Shield |
| 162 | Bridges |
| 170 | Proximal End |
| 172 | Long Ribbons |
| 174 | Short Ribbons |
| 176 | Connector Pads |
| 178 | Fish Hole |
| 182 | Cut Tab |
| 200 | Flexible Circuit |
| 205 | Top Cover Layer |
| 208 | Ground Bar |
| 210 | Distal End |
| 212 | Plurality of Traces |
| 213 | Upper Region |
| 214 | Transducer Attachment |
| 230 | Signal Ribbons |
| 230A | First Signal Ribbon |
| 230B | Second Signal Ribbon |
| 230C | Third Signal Ribbon |
| 230D | Fourth Signal Ribbon |
| 234 | Horizontal Bar |
| 238 | Space |
| 250 | Middle Region |
| 256 | Ground Shield |
| 258 | Two Panel Shield |
| 260 | Three Panel Shield |

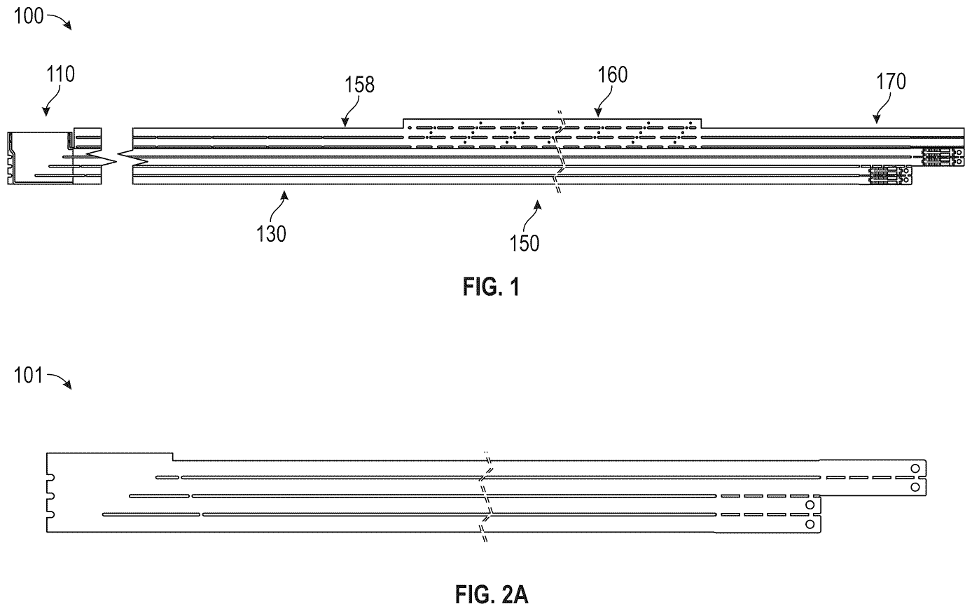

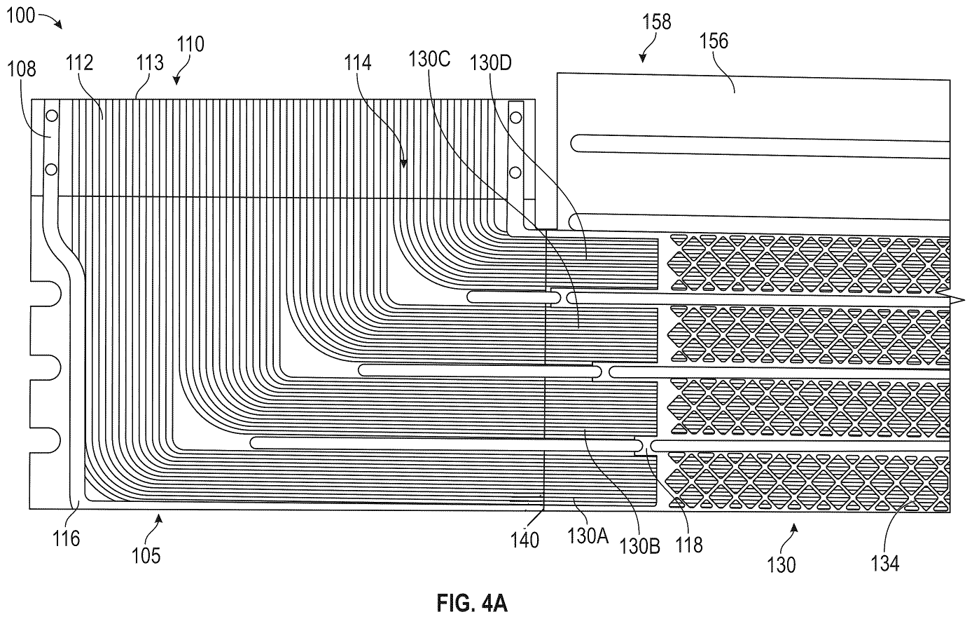

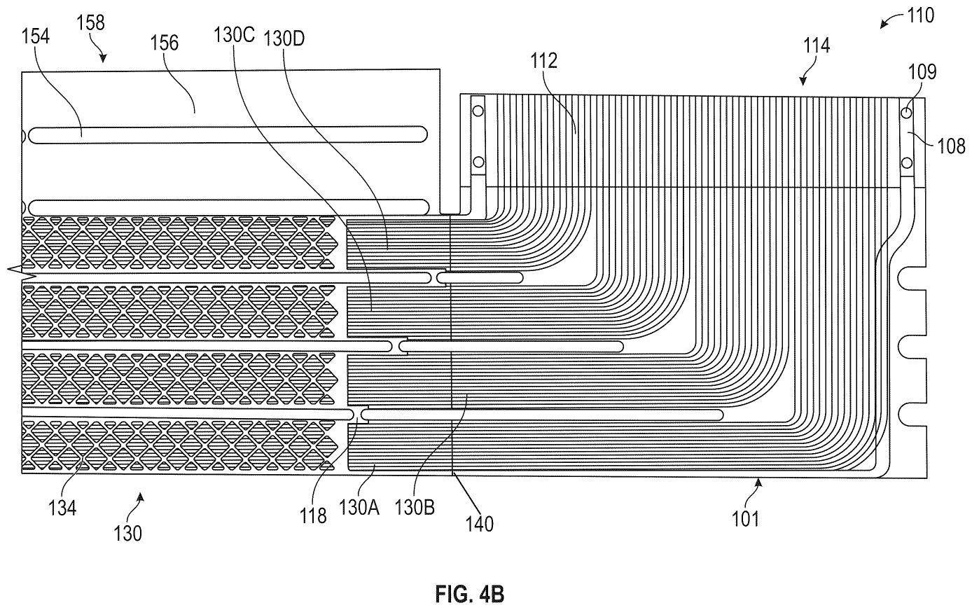

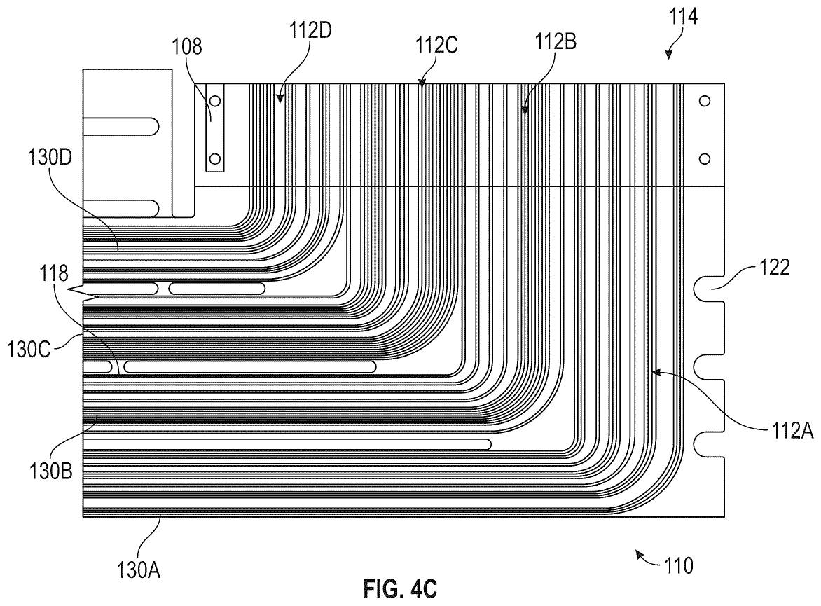

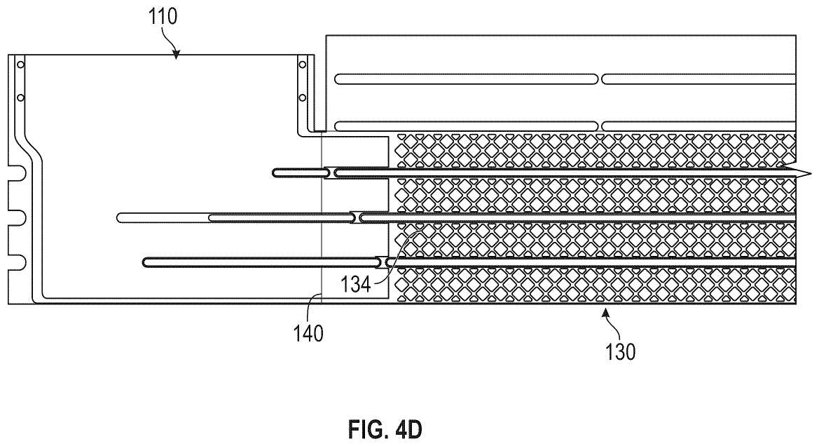

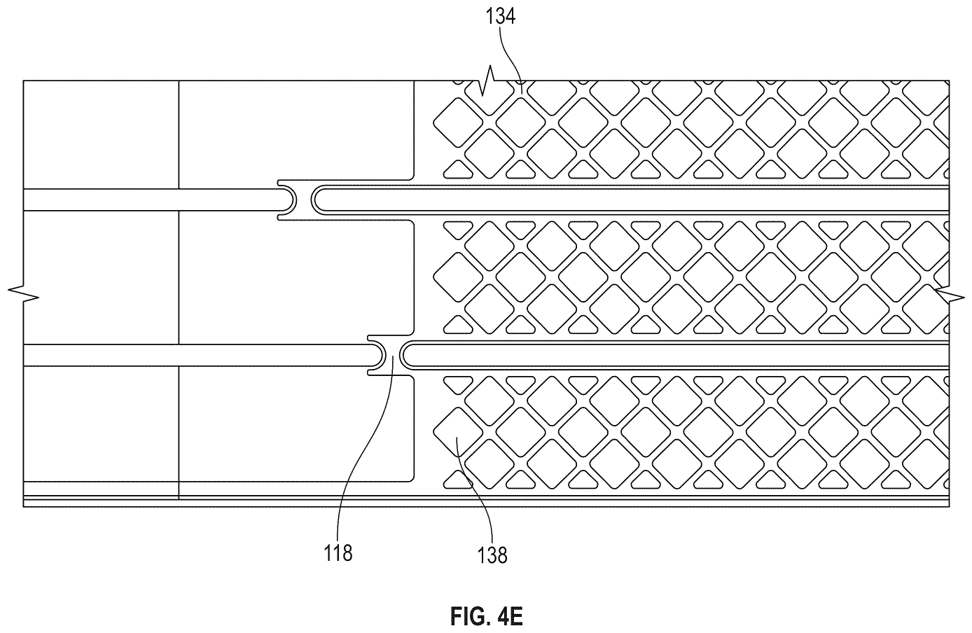

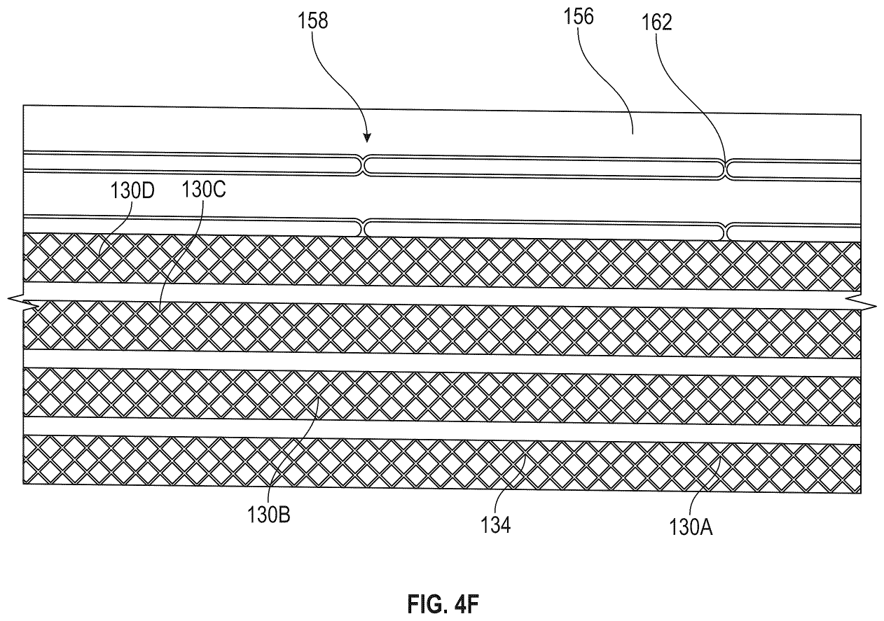

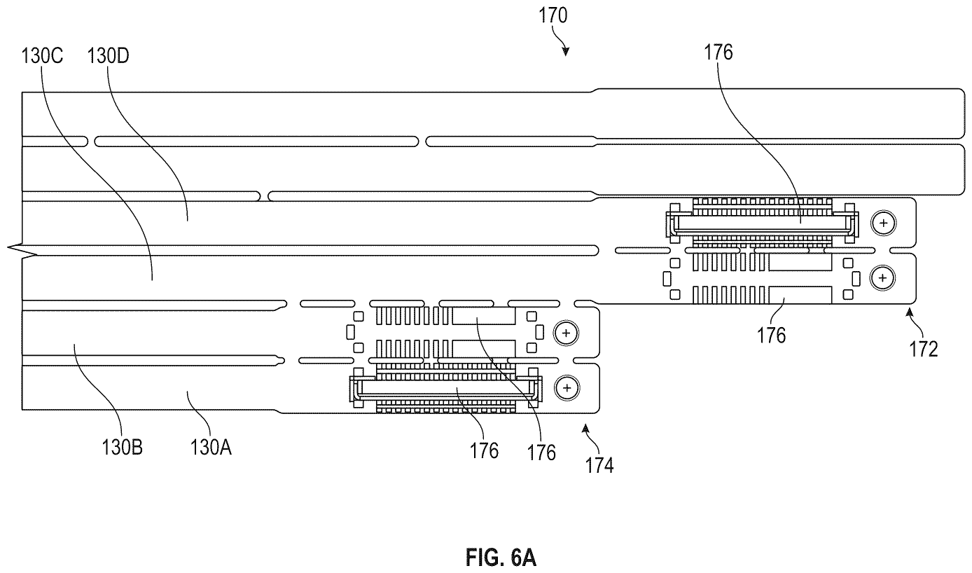

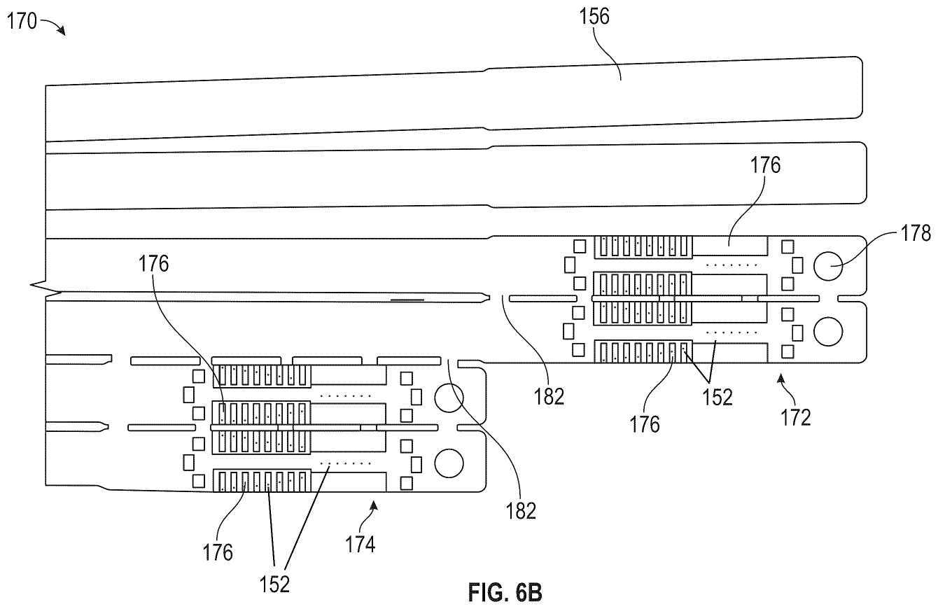

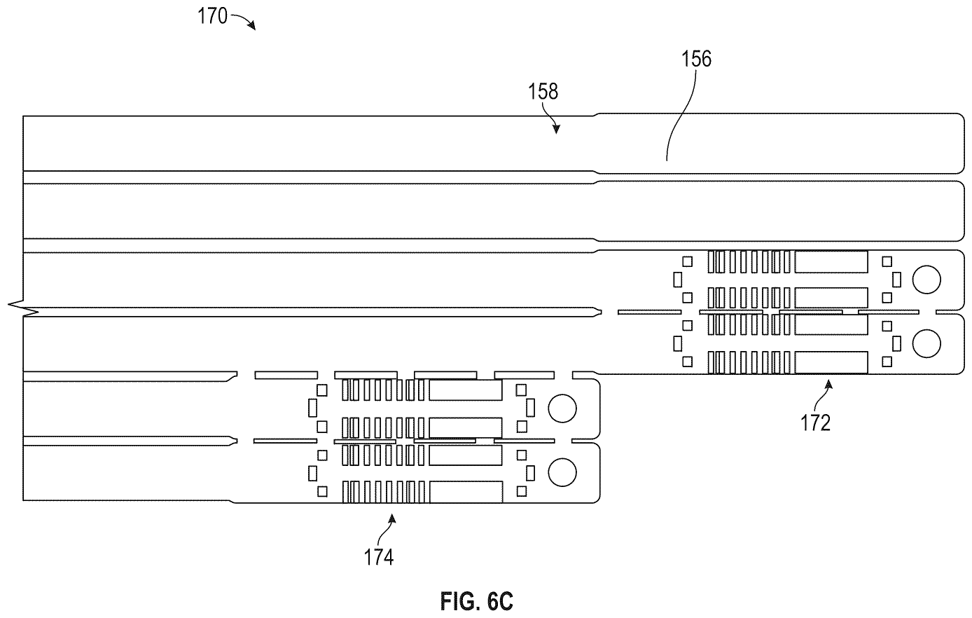

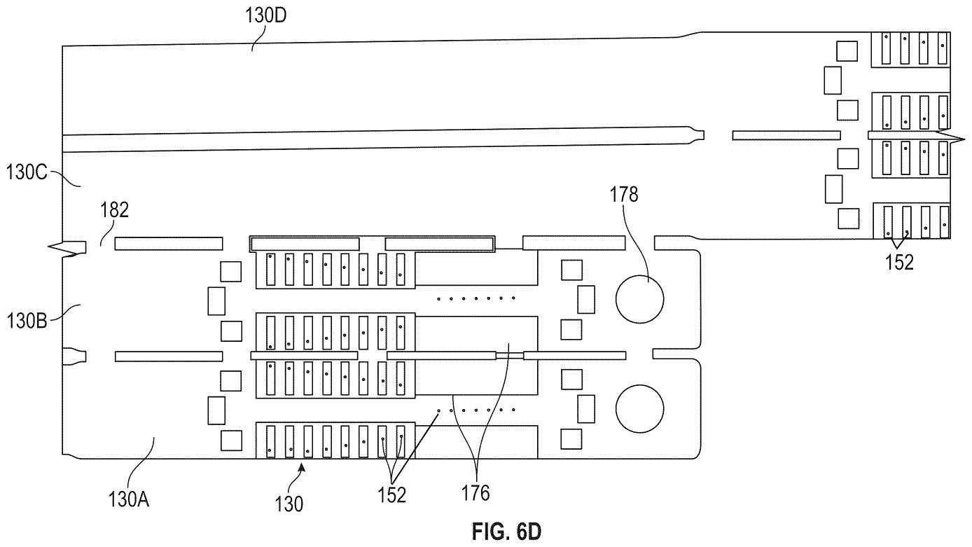

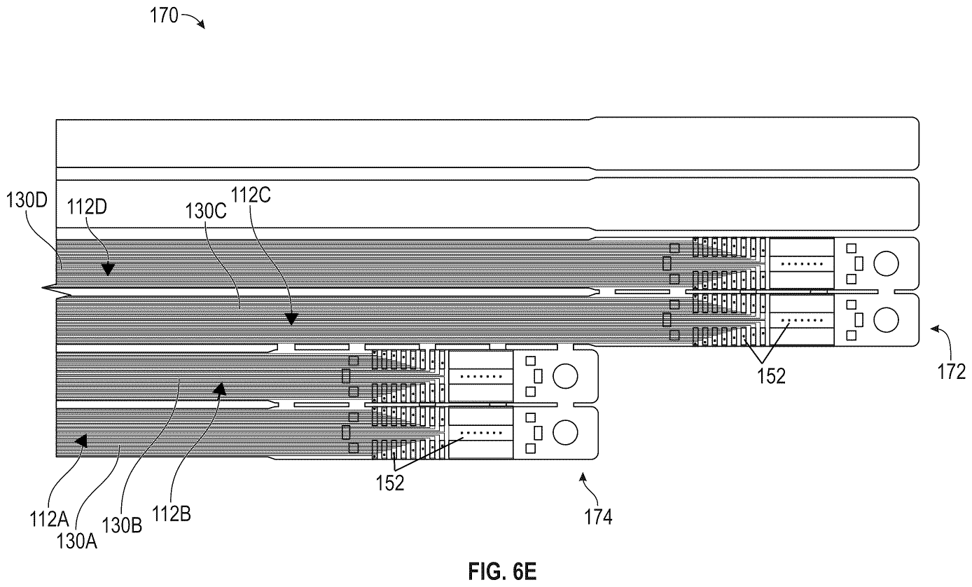

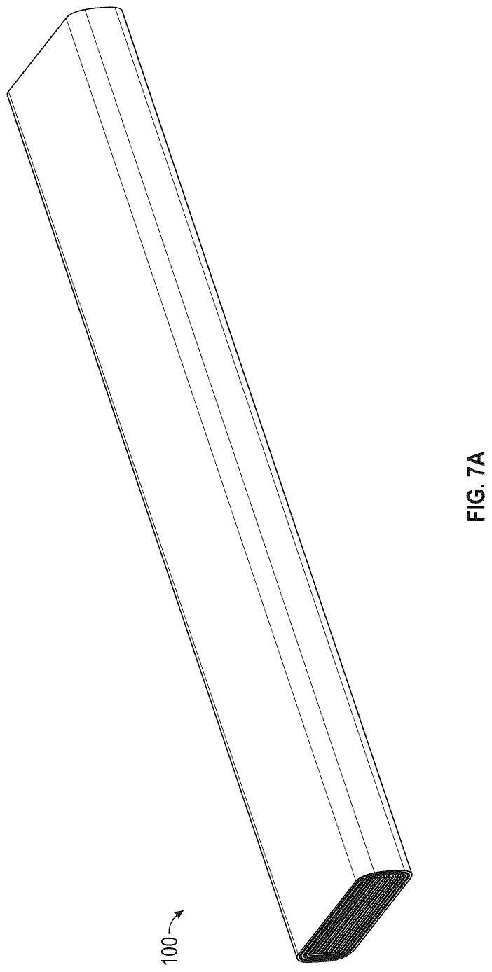

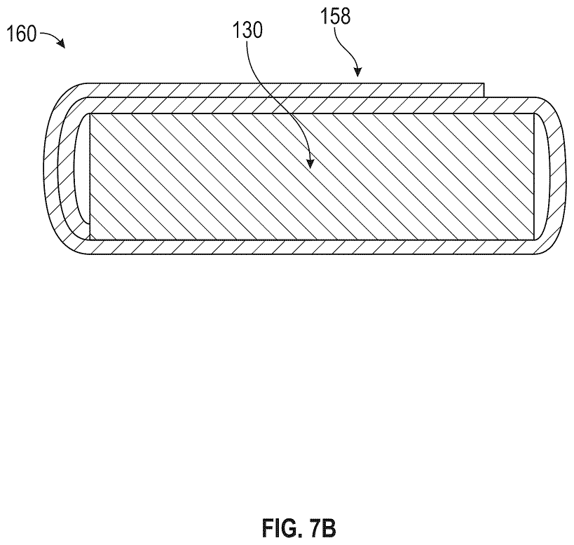

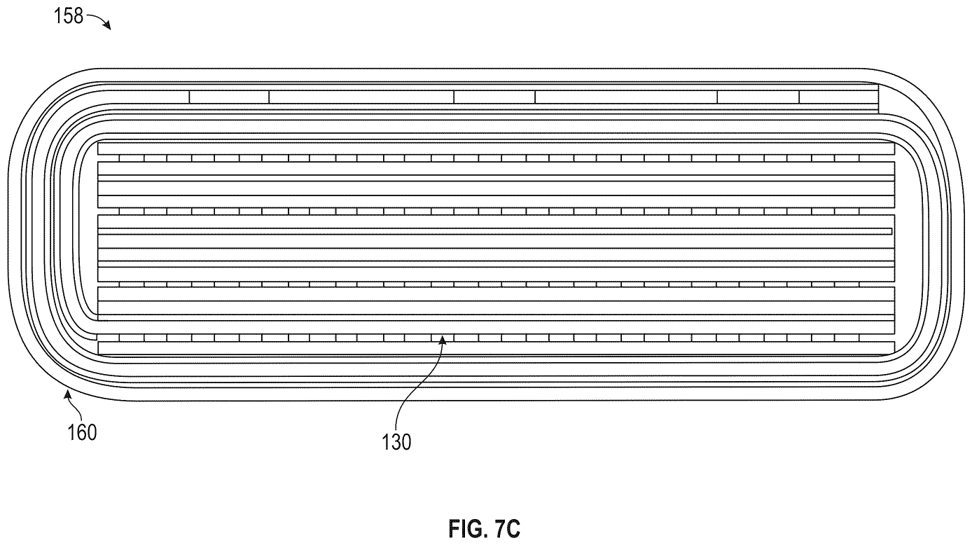

FIG. 1 shows a top view of an example of a flexible circuit 100 which can be used in an intracardiac echocardiography (“ICE”) catheter device in a flat (e.g., unfolded) state. The flexible circuit 100 as shown in FIG. 1 can include a distal end 110 or distal region, a plurality of signal ribbons 130 (e.g., four signal ribbons), a middle region 150 or medial region, and a proximal end 170 or proximal region. As used herein, the terms “proximal end” and “distal end” are used to refer to the relative positions on a circuit (e.g., flexible circuit 100) or a device. With regard to the flexible circuit 100 shown in FIG. 1, for example, the distal end 110 is the end closest to the transducer attachment 114 (see FIG. 4A), while the proximal end 170 is the end closest to connector pads 176 or sensing elements (e.g., small, conductive surface areas) (see FIG. 6A). The middle region 150 can include a two panel shield section 158 (e.g., first shielding section) and a three panel shield section 160 (e.g., second shielding section). The three panel shield section 160 can have three copper or ground shields 156. The two panel shield section can have two copper or ground shields 156. In some embodiments, the flexible circuit 100 can have a total length (e.g., the length from the end of the distal end 110 to the end of the proximal end 170) of approximately 1124 millimeters. In other embodiments, the flexible circuit 100 can have a length between, or about, 1000 millimeters, 1020 millimeters, 1040 millimeters, 1060 millimeters, 1080 millimeters, 1100 millimeters, 1140 millimeters, 1160 millimeters, 1180 millimeters, 1200 millimeters or 1220 millimeters. The flexible circuit 100 is foldable such that the plurality of signal ribbons 130 can positioned over one another in order to be assembled into a catheter device (see for example, FIGS. 7A-7C which illustrates folded configurations).

As used herein, “flexible” can generally refer to the ability (e.g., of a material or an article) to bend freely and repeatably without breaking and/or to conform to the shape of the body part to which the flexible material (or article) is applied. The length from transducer attachment 114 extending from the distal end 110 to the distal most end of the three panel shield section 160 can have a length of approximately one hundred and one millimeters. Additionally, the length from transducer attachment 114 extending from the distal end 110 to the distal most end of the three panel shield section 160 can have a length of approximately 90 millimeters, 95 millimeters, one hundred and five millimeters, and/or one hundred and ten millimeters. The distance from the one or more connector pads 176 extending from the proximal end of the short ribbons 174 to the proximal end of the three panel shield section 160 can have a length of approximately 40 millimeters. Additionally, the distance from the one or more connector pads 176 extending from the proximal end of the short ribbons 174 to the proximal end of the three panel shield section 160 can have a length of approximately 36 millimeters, 37 millimeters, 38 millimeters, 39 millimeters, 41 millimeters, 42 millimeters, 43 millimeters, and/or 44 millimeters. The distance from the one or more connector pads 176 extending from the proximal end of the set of long ribbons 172 to the proximal end of the three panel shield section 160 can have a length of approximately 50 millimeters. Additionally, the distance from the one or more connector pads 176 extending from the proximal end of the short ribbons 174 to the proximal end of the three panel shield section 160 can have a length of approximately 45 millimeters, 46 millimeters, 47 millimeters, 48 millimeters, 49 millimeters, 51 millimeters, 52 millimeters, 53 millimeters, 54 millimeters, or 55 millimeters. The distance from the proximal end of the three panel shield section 160 to the distal end of the three panel shield section 160 can have a length of approximately 972 millimeters. Additionally, The distance from the proximal end of the three panel shield section 160 to the distal end of the three panel shield section 160 can have a length of 870 millimeters, 900 millimeters, 930 millimeters, 960 millimeters, 990 millimeters, 1020 millimeters, 1050 millimeters, or 1080 millimeters.

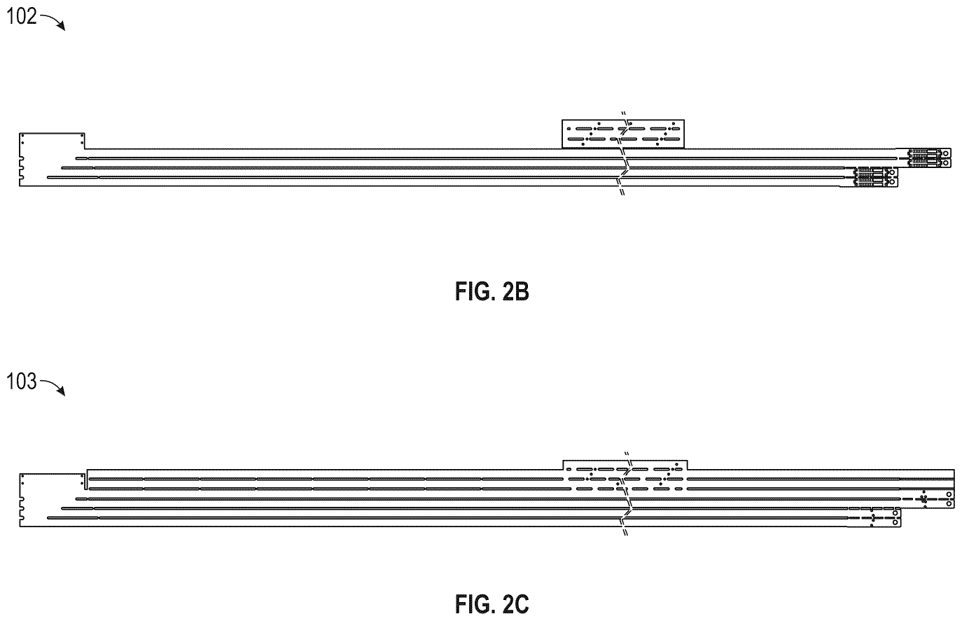

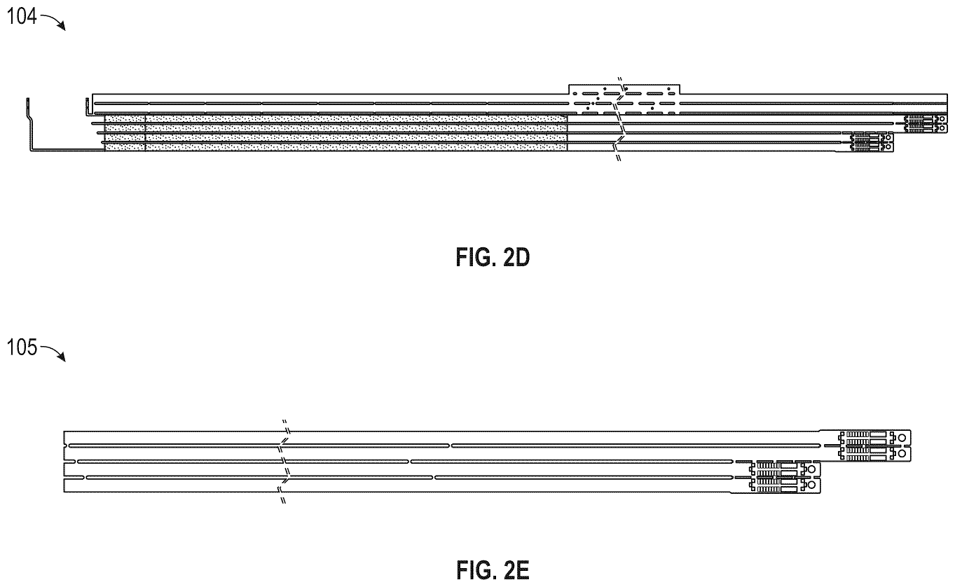

FIGS. 2A-2E illustrate a plurality of layers of the flexible circuit 100 which can include a bottom cover layer 101, a bottom ground (e.g., copper) layer 102, a base layer 103 (e.g., polyimide), a top ground (e.g., copper) layer 104, and a top cover layer 105. FIG. 2A illustrates the bottom cover layer 101. FIG. 2B illustrates the bottom ground layer 102. FIG. 2C illustrates the base layer 103. FIG. 2D illustrates the top ground layer 104. FIG. 2E illustrates the top cover layer 105. The plurality of layers (e.g., bottom cover layer 101, bottom ground layer 102, base layer 103, top ground layer 104, and top cover layer 105) can be placed on top of one another in order to form the flexible circuit 100. The top cover layer 105 can provide additional stiffness to the flexible circuit 100 in order to protect the electronic components of the flexible circuit 100 (e.g., the plurality of traces 112).

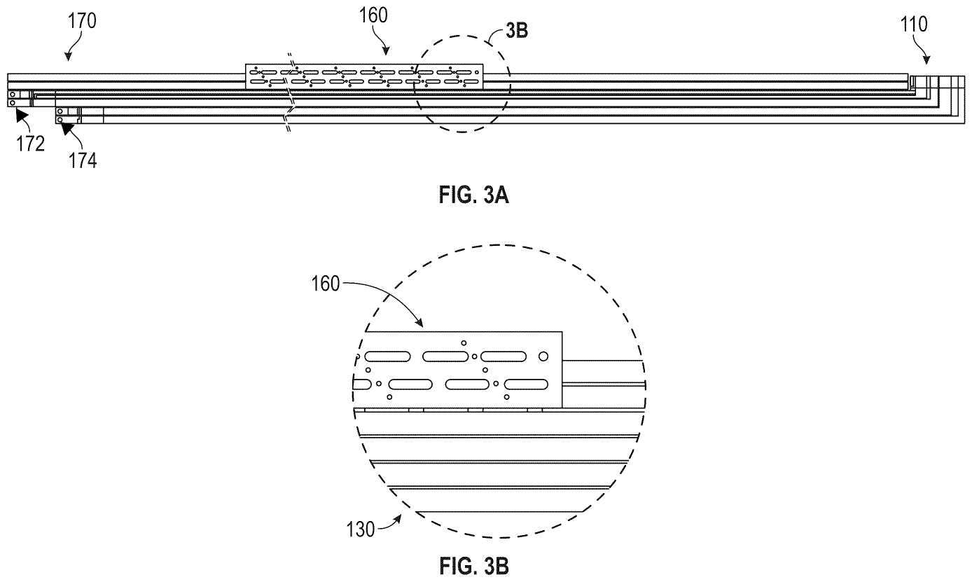

FIG. 3A illustrates a bottom view of flexible circuit 100 (e.g., where the bottom cover layer 101 is shown). FIG. 3B illustrates a detailed view of the middle region 150 of the flexible circuit 100. The three panel shield section 160 can be exposed when the flexible circuit 100 is viewed from the rear or bottom perspective. Additionally, the three panel shield section 160 and the two panel shield section 158 can be positioned above the top cover layer 105 when the layers of the flexible circuit 100 are stacked on top of one another and the flexible circuit 100 is in the unfolded configuration. Therefore, in some embodiments, the three panel shield section 160 and the two panel shield section 158 are positioned such that they can fold over the plurality of signal ribbons 130 in order to place the flexible circuit 100 in a folded state. The plurality of signal ribbons 130 can have a set of long ribbons 172 and a set of short ribbons 174 (e.g., the set of long ribbons 172 can extend beyond the set of short ribbons 174). The set of long ribbons 172 can be two ribbons. The set of long ribbons 172 can also be, for example, three, four, or five ribbons. The set of short ribbons 174 can be two ribbons. The set of short ribbons 174 can also be, for example, three, four or five ribbons. At the proximal end 170 the set of short ribbons 174 can terminate at a position distal to the set of long ribbons 172.

The total height of the three panel shield section 160 can be approximately 5 millimeters. Additionally, the total height of the three panel shield section 160 can be approximately 2.25 millimeters, 3 millimeters, 3.75 millimeters, 4.25 millimeters, 4 millimeters, or 4.5 millimeters. The total height of the plurality of signal ribbons 130 and the three panel shield section 160 is approximately 12 millimeters. Additionally, the total height of the plurality of signal ribbons 130 and the three panel shield section 160 can be approximately 9 millimeters, 10 millimeters, 11 millimeters, 13 millimeters, 14 millimeters, or 15 millimeters. The set of long ribbons 172 can have a length extending from the proximal end 170 to the proximal most end of the transducer attachment 114 of approximately 1124 millimeters. Additionally, the set of long ribbons 172 can have a length extending from the proximal end 170 to the proximal most end of the transducer attachment 114 of approximately 1000 millimeters, 1020 millimeters, 1040 millimeters, 1060 millimeters, 1080 millimeters, 1100 millimeters, 1140 millimeters, 1160 millimeters, 1180 millimeters, 1200 millimeters or 1220 millimeters. The set of short ribbons 174 can have a length extending from the proximal end 170 to the proximal most edge of the transducer attachment 114 of approximately 1114 millimeters. Additionally, the length of the set of short ribbons 174 extending from the proximal end 170 to the proximal most edge of the transducer attachment 114 can be approximately 990 millimeters, 1110 millimeters, 1130 millimeters, 1150 millimeters, 1170 millimeters, 1190 millimeters, 1210 millimeters.

FIGS. 4A-4F illustrate the distal end 110 of the flexible circuit 100. The distal end 110 can include a flexible region. The distal end 110 can have a transducer attachment 114. The length of the transducer attachment 114 extending from the distal end 110 to the beginning portion (e.g., the beginning portion being adjacent to the coverlay region 140) of the two panel shield section 158 can have a length of approximately 12.3 millimeters. The length of the transducer attachment 114 extending from the distal end 110 to the beginning portion of the two panel shield section 158 can have a length of approximately 11 millimeters, 11.5 millimeters, 12 millimeters, 12.5 millimeters, 13 millimeters, 13.5 millimeters, or 14 millimeters. The transducer attachment 114 can include a plurality of traces 112. The plurality of traces 112 can extend from a top or upper region 113 of the transducer attachment 114 to the plurality of signal ribbons 130 and terminate at the proximal end 170 (see FIGS. 6A-6E). Each trace of the plurality of traces 112 can be terminated by a 32-pin connector. Additionally, in some embodiments, half or approximately half of the plurality of traces 112 are electrically coupled to a ground portion or the ground bar 108. The transducer attachment 114 can be positioned adjacent to (e.g., horizontal) the plurality of signal ribbons 130. The plurality of signal ribbons 130 can extend from the transducer attachment 114 at a position near or adjacent to an endpoint of the coverlay region 140. The transducer attachment 114 can have a ground section or ground bar 108 which can be positioned along an outside edge 116 of the transducer attachment 114. The top or upper region 113 can be approximately twelve millimeters. Additionally, the top or upper region 113 can be approximately nine millimeters, ten millimeters, eleven millimeters, thirteen millimeters, or fourteen millimeters. The outside edge 116 at a distalmost region can be approximately ten millimeters. Additionally, the outside edge 116 can be approximately eight millimeters, nine millimeters, eleven millimeters, twelve millimeters, or thirteen millimeters.

The plurality of traces 112 can include sixty-four traces. The plurality of traces 112 can also be sixty traces, fifty-eight traces, fifty traces, forty traces, or thirty-two traces. The plurality of traces 112 can extend from the top or upper region 113 and split into multiple sets (e.g., four sets). Each set of the plurality of traces 112 can extend into a signal ribbon of the plurality of signal ribbons 130. For example, the plurality of traces 112 can split into four sets of traces (e.g., each set can have sixteen traces) and each set (e.g., first traces 112A, second traces 112B, third traces 112C, and fourth traces 112D) of the of the plurality of traces 112 can extend into one signal ribbon of the plurality of signal ribbons 130. The plurality of signal ribbons 130 can be four signal ribbons, which can include a first signal ribbon 130A, a second signal ribbon 130B, a third signal ribbon 130C, and a fourth signal ribbon 130D. The plurality of signal ribbons 130 can bend or flex during operation. The plurality of traces 112 can be spaced apart by approximately 0.08 millimeters. Additionally, the plurality of traces 112 can be spaced apart approximately 0.16 millimeters.

The plurality of traces 112 can be positioned on the bottom ground layer 102. Additionally, the transducer attachment 114 can be positioned on the bottom ground layer 102. The plurality of signal ribbons 130 can have a width of approximately 1.5 millimeters. Advantageously, the width of the plurality of signal ribbons 130 can fit inside a 9 French catheter shaft. Additionally, a width of a signal ribbon of the plurality of signal ribbons 130 can be approximately 0.75 millimeters, one millimeter, 1.25 millimeters, 1.75 millimeters, millimeters, or 2.25 millimeters. The total width of the plurality of signal ribbons 130 (e.g., including the first signal ribbon 130A, the second signal ribbon 130B, the third signal ribbon 130C, and the fourth signal ribbon 130D) can be approximately 6 millimeters. Additionally, the total width of the plurality of signal ribbons 130 can be approximately 3 millimeters, 4 millimeters, 5 millimeters, 7 millimeters, 8 millimeters, or 9 millimeters. The plurality of signal ribbons 130 can be connected to each other with one or more tabs 118. The plurality of signal ribbons 130 can fold on top of one another at the one or more tabs 118. Additionally, the one or more tabs 118 can be cut or broken to ease the folding of the plurality of signal ribbons 130 to place the flexible circuit 100 in the folded configuration (see FIG. 7A). The distal end 110 can include one or more cutouts 122. The one or more cutouts 122 can be spaced vertically at the distal end 110 and can ease folding the plurality of signal ribbons 130 when converting the flexible circuit 100 from the unfolded configuration to the folded (e.g., wrapped) configuration.

The plurality of signal ribbons 130 extending from the transducer attachment 114 at the end of the coverlay region 140 can have a crosshatch 134. The crosshatch 134 can improve the flexibility of the plurality of signal ribbons 130 at the distal end 110. The crosshatch 134 can connect ground regions between the plurality of signal ribbons 130 when the flexible circuit 100 is in the folded configuration. The crosshatch 134 can be positioned on the top ground layer 104. The crosshatch 134 can be positioned on or adjacent to the top cover layer 105. Additionally, the crosshatch 134 can be operably connected to (e.g., physically or electrically coupled) the ground bar 108. The crosshatch 134 can have a grid-like pattern which can advantageously balance the amount of ground material along the plurality of signal ribbons 130 with a desired flexibility of the plurality of signal ribbons 130. The crosshatch 134 can have a plurality of gaps 138 positioned along the plurality of signal ribbons 130 at the distal end 110 or flex region (e.g., the portion of the plurality of signal ribbons 130 adjacent to the transducer attachment 114). The plurality of gaps 138 can be approximately 0.5 millimeters in length or width. The plurality of gaps 138 can be approximately 0.35 millimeters, 0.4 millimeters, 0.45 millimeters, 0.55 millimeters, 0.6 millimeters in length or width. The plurality of gaps 138 can provide additional flexibility for the plurality of signal ribbons 130.

The two panel shield section 158 can start (e.g., extend from the distal end 110 to the proximal end 170) at the coverlay region 140. The two panel shield section 158 can be adjacent (e.g., above) to the fourth signal ribbon 130D in the unfolded configuration. For example, one ground shield 156 of the two panel shield section 158 (e.g., shield 156A) can be positioned above the fourth signal ribbon 130D. A second ground shield 156B of the two panel shield section 158 can be positioned above the first ground shield 156A. The two panel shield section 158 can protect or cover the plurality of signal ribbons 130 when flexible circuit 100 is in the folded configuration. The ground shields 156 can be spaced apart and at least partially separated from each other with one or more holes, perforations, or slots 154. The slots 154 can be approximately fifteen millimeters in length. Additionally, the slots 154 can be approximately ten millimeters in length. The slots 154 can be approximately three millimeters in length. Advantageously, the slots 154 can prevent the plurality of signal ribbons 130 from bunching (e.g., bulging) when the flexible circuit 100 is in the folded configuration and when the flexible circuit 100 is bent during operation (e.g., during insertion into a patient).

The slots 154 can terminate (e.g., end) at bridges 162. The bridges 162 can connect the fourth signal ribbon 130D to a first ground shield 156 of the two panel shield section 158. Additionally, the bridges 162 can connect the first ground shield 156 and the second ground shield 156 in the two panel shield section 158. In some regions (e.g., the distal end 110 and/or flex region) the bridges 162 can be stacked above each other. In some embodiments, the bridges 162 can be staggered. Advantageously, staggering the bridges 162 can allow the flexible circuit 100 to lay flat (e.g., flatter then in a stacked orientation) when the flexible circuit 100 is in the folded configuration.

The length of the transducer attachment 114 extending from the distal end 110 to the beginning portion of the plurality of signal ribbons 130 can have a length of 12 millimeters. The ground bar 108 can have at least one hole 109. The distance between the top of the ground bar 108 and the at least one hole 109 can be approximately 0.4 millimeters. The distance between a first at least one hole 109 on the ground bar and a second at least one hole 109 on the ground bar can be approximately 1.7 millimeters. The distance between the edge of the distal end 110 and the ground bar 108 can be approximately 0.45 millimeters. The distance between the ground bar 108 and the nearest of the plurality of traces 112 can be approximately 0.27 millimeters. Additionally, the length of the transducer attachment 114 extending from the distal end 110 to the beginning portion of the plurality of signal ribbons 130 can have a length of approximately 11 millimeters, 11.5 millimeters, 12.5 millimeters, or 13 millimeters. In some embodiments, the distance between the top of the ground bar 108 and the at least one hole can be approximately 0.35 millimeters, 0.36 millimeters, 0.37 millimeters, 0.38 millimeters, 0.39 millimeters, 0.41 millimeters, 0.42 millimeters, 0.43 millimeters, 0.44 millimeters, or 0.45 millimeters. In some embodiments, the distance between a first at least one hole 109 and a second at least one hole 109 can be approximately 1.55 millimeters, 1.6 millimeters, 1.65 millimeters, 1.75 millimeters, 1.8 millimeters, or 1.85 millimeters. In some embodiments, the distance between the edge of the distal end 110 and the ground bar 108 can be 0.4 millimeters, 0.41 millimeters, 0.42 millimeters, 0.43 millimeters, 0.44 millimeters, 0.46 millimeters, 0.47 millimeters, 0.48 millimeters, 0.49 millimeters, or 0.5 millimeters. Additionally, the width of the ground bar 108 can be approximately 0.4 millimeters. Additionally, the width of the ground bar 108 can be approximately 0.375 millimeters, 0.38 millimeters, 0.385 millimeters, 0.39 millimeters, 0.395 millimeters, 0.405 millimeters, 0.41 millimeters, 0.415 millimeters, 0.42 millimeters, 0.425 millimeters. Additionally, the distance between the ground bar 108 and the nearest of the plurality of traces 112 can be approximately 0.25 millimeters, 0.26 millimeters, 0.28 millimeters, or 0.29 millimeters.

The one or more cutouts 122 can have a depth of approximately 0.4 millimeters. The one or more cutouts can have a height of approximately 0.58 millimeters. The lowest of the one or more cutouts 122, can have its center approximately 1.6 millimeters above the bottom of the distal end 110. The highest of the one or more cutouts 122, can have its center approximately 5 millimeters above the bottom of the distal end 110.

In some embodiments, the total depth of the one or more cutouts 122 can be approximately 0.35 millimeters, or 0.45 millimeters. In some embodiments, the total height of the one or more cutouts 122 can be approximately 0.5 millimeters, 0.55 millimeters, 0.6 millimeters, or 0.65 millimeters. In some embodiments, the lowest of the one or more cutouts 122, can have its center approximately be 1.45 millimeters above the bottom of the distal end 110, 1.50 millimeters above the bottom of the distal end 110, 1.55 millimeters above the bottom of the distal end 110, 1.65 millimeters about the bottom of the distal end, 1.70 millimeters above the bottom of the distal end 110, or 1.75 millimeters above the bottom of the distal end 110. In some embodiments, the highest of the one or more cutouts 122, can have its center approximately 4.5 millimeters above the bottom of the distal end 110, 4.6 millimeters above the bottom of the distal end 110, 4.7 millimeters above the bottom of the distal end, 4.8 millimeters above the bottom of the distal end 110, 4.9 millimeters above the bottom of the distal end 110, 5.1 millimeters above the bottom of the distal end 110, 5.2 millimeters above the bottom of the distal end 110, 5.3 millimeters above the bottom of the distal end 110, 5.4 millimeters above the bottom of the distal end 110, or 5.5 millimeters above the bottom of the distal end 110.

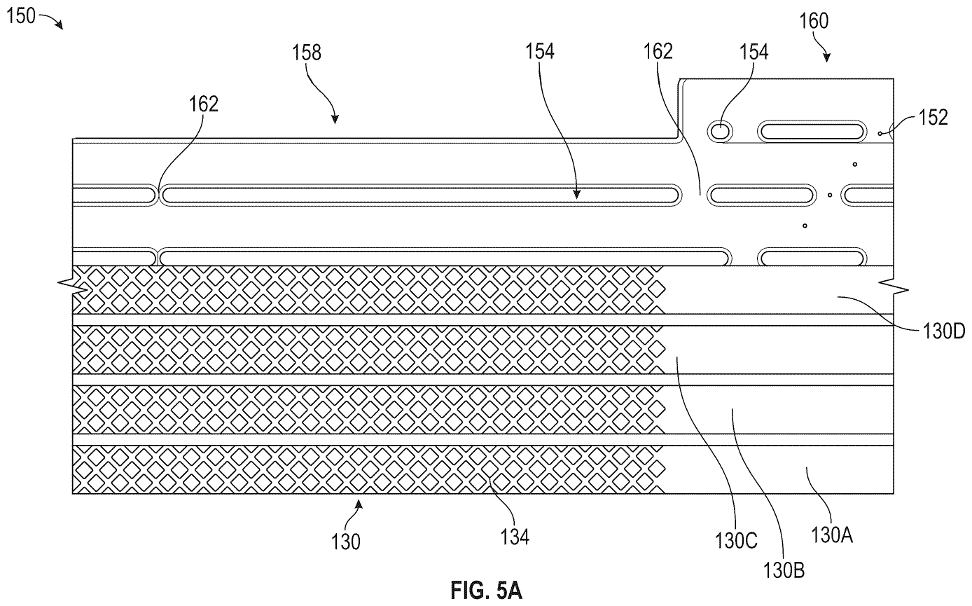

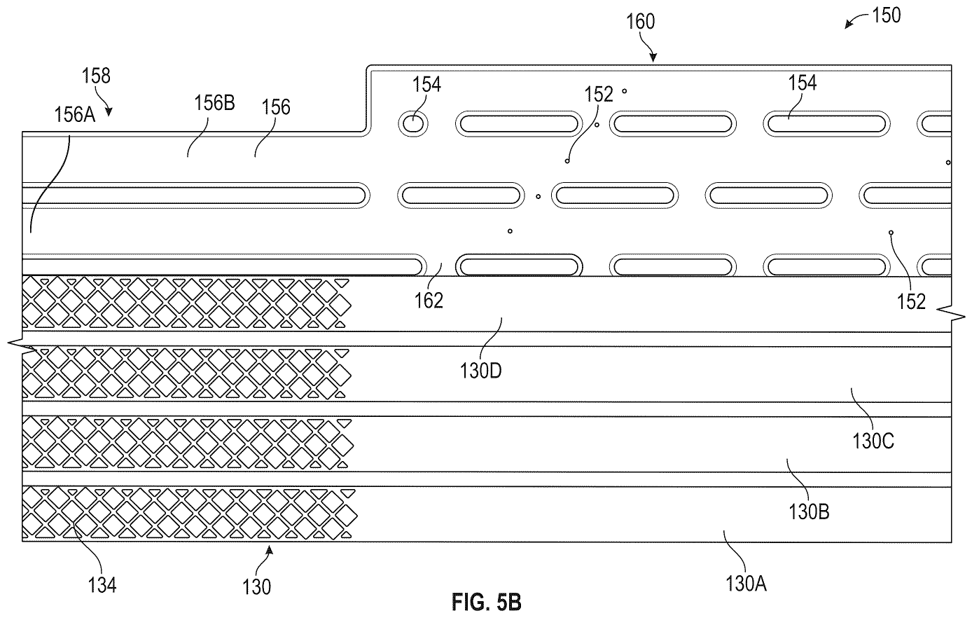

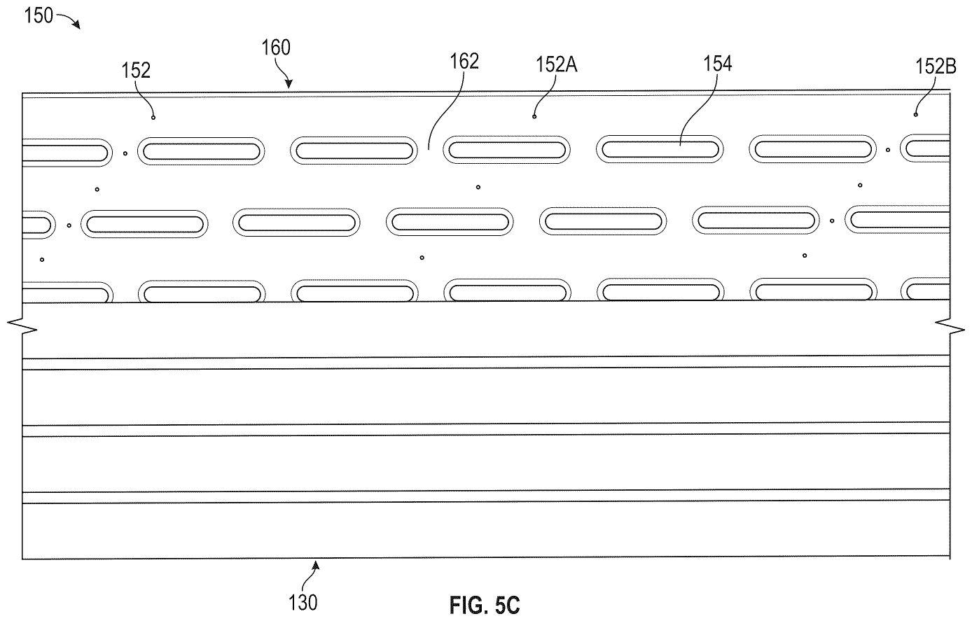

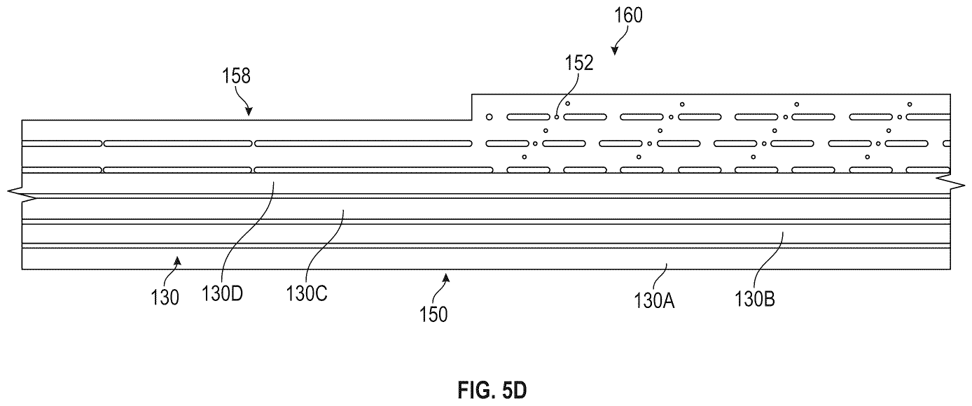

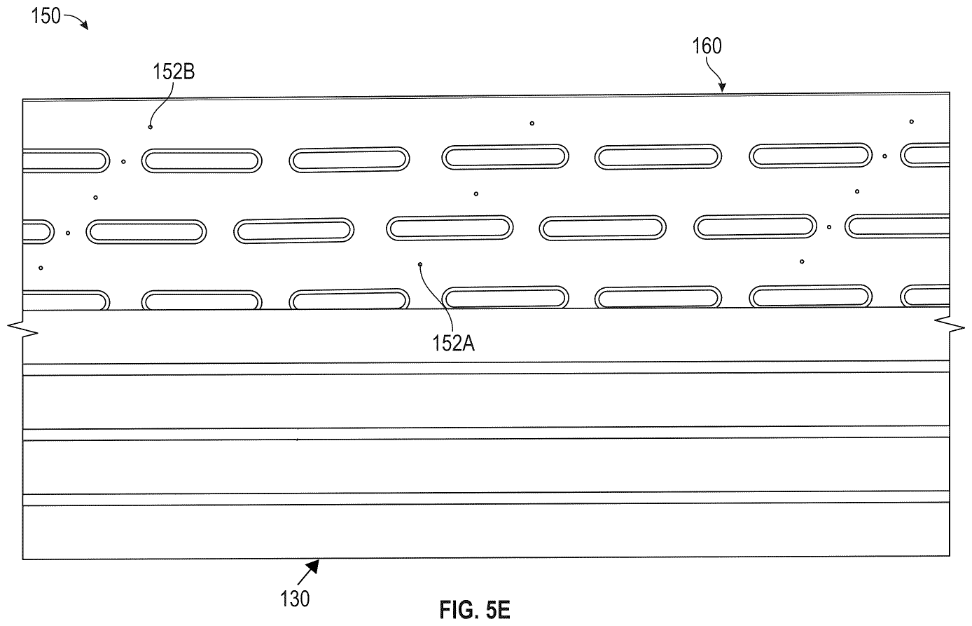

FIGS. 5A-5E illustrate a middle region 150 of the flexible circuit 100. The plurality of signal ribbons 130 extend from the distal end 110 at the transducer attachment 114 and through the middle region 150. The crosshatch 134 can terminate (e.g., end) in the middle region 150. The middle region 150 can include a three panel shield section 160. The three panel shield section 160 can include three ground shields 156 positioned adjacent (e.g., one ground shield 156 above the other). The three panel shield section 160 can be positioned above the plurality of signal ribbons 130 (e.g., above the fourth signal ribbon 130D). The three panel shield section 160 can be mechanically coupled to the fourth signal ribbon 130D with the bridges 162. The bridges in the three panel shield section 160 can be staggered (e.g., extend along a diagonal). Additionally, the three panel shield section 160 can have slots 154 which are positioned along the length of the three panel shield section 160. The slots 154 can be approximately three 3 millimeters in length. In some embodiments, the length of the three panel shield section 160 is approximately 972 millimeters. Each of the ground shields 156 in the three panel shield section 160 (or two panel shield section 158) can have a width of approximately 1.5 millimeters. Advantageously, the width of the one ground shield 156 can correspond to a width of one of the plurality of signal ribbons 130. In some embodiments, the slots 154 can be approximately 0.4 millimeters tall. The slots 154 can be approximately 0.3 millimeters tall, 0.35 millimeters tall, 0.45 millimeters tall, or 0.5 millimeters tall. Additionally, the slots 154 can be approximately 1.00 millimeter apart horizontally. The slots 154, can be approximately 0.5 millimeters apart horizontally, 1.5 millimeters apart horizontally, 2.0 millimeters apart horizontally, or 2.5 millimeters apart horizontally. In some embodiments, the length of the three panel shield section 160 can be approximately a length of 900 millimeters, 910 millimeters, 930 millimeters, 940 millimeters, 950 millimeters, 960 millimeters, 980 millimeters, 990 millimeters, 1000 millimeters, 1010 millimeters, 1020 millimeters, 1030 millimeters, or 1040 millimeters.

The three panel shield section 160 can include a plurality of vias 152 positioned along the length of the ground shield 156. The plurality of vias 152 can be 0.10 millimeters in size. Additionally, the plurality of vias 152 can be approximately 0.09 millimeters in size, 0.095 millimeters in size, or 0.15 millimeters in size. The plurality of vias 152 can be positioned on or near a middle area of the ground shield 156. The plurality of vias 152 can connect ground regions across the layers of the flexible circuit 100 (e.g., bottom ground layer 102, base layer 103, top ground layer 104). For example, the plurality of vias 152 can stitch the three panel shield section 160 to a ground portion of the bottom ground layer 102. The plurality of vias 152 can connect ground regions across the plurality of signal ribbons 130. Advantageously, when the flexible circuit 100 is in a folded configuration, the plurality of vias 152 can be optimally spaced along the length of the three panel shield section 160 in order to maintain reliable connections (e.g., electrical connections).

The three panel shield section 160 can stagger the plurality of vias 152. For example, the plurality of vias 152 can be staggered (e.g., extend diagonally along the length of the three panel shield section 160) and have two different regions. The plurality of vias 152 can be spaced apart approximately 2.3 millimeters from each other in a vertical direction. The plurality of vias 152 can have a set of three vias 152A and a set of five vias 152B. Advantageously, having a set of three vias 152A and a set of five vias 152B can balance the flexibility of the three panel shield section 160 while providing connections (e.g., electrical connections) between the plurality of signal ribbons 130 and the flexible circuit 100 when in a folded position. The set of three vias 152A and the set of five vias 152B can be approximately 7 millimeters apart from the proximal most via on the set of five vias 152B to the distal most via on the set of three vias. The set of three vias 152A and the set of five vias 152B can be approximately 10 millimeters from the distal most via on the set of five vias 152B and the proximal via on the set of three vias 152A. In some embodiments, the set of three vias 152A and the set of five vias 152B can be approximately six millimeters, eight millimeters, nine millimeters, or ten millimeters apart from the proximal most via on the set of five vias 152B to the distal most via on the set of three vias 152A. The plurality of vias 152 can be spaced approximately 22 millimeters from opposing ends of the three panel shield section 160.

FIGS. 6A-6E illustrate the proximal end 170 of the flexible circuit 100. The plurality of traces 112 extending from the distal end 110 and through the middle region 150 can terminate at the proximal end 170. The plurality of traces 112 can terminate at one or more connector pads 176 positioned at the end of the plurality of signal ribbons 130. For example, the first traces 112A, second traces 112B, third traces 112C, and fourth traces 112D can terminate at a corresponding connector pad 176. The one or more connector pads 176 can facilitate connections to a plurality of sensing elements or hardware (e.g., sensing chips, processing chips, etc.). The one or more connector pads 176 can have a plurality of vias 152 (e.g., a third set of vias) extending along the length of the one or more connector pads 176. The plurality of vias 152 can be positioned on the top cover layer 105. The plurality of vias 152 can pass through the layers (e.g., bottom cover layer 101, bottom ground layer 102, base layer 103, top ground layer 104) of the flexible circuit 100. Additionally, the plurality of traces 112 can terminate at corresponding locations along the length of the one or more connector pads 176 to ease assembly and to reduce the chance of damaging the plurality of traces 112 during assembly. The plurality of signal ribbons 130 can have a set of long ribbons 172 and a set of short ribbons 174. Advantageously, the difference in length between the set of long ribbons 172 and the set of short ribbons 174 can account for a staggered landing position of the one or more connector pads 176 on a connection port. Having the set of long ribbons 172 and the set of short ribbons 174 can reduce a need to fold and tuck the excess length of the plurality of signal ribbons 130 when the flexible circuit 100 is in the folded configuration. Additionally, having set of long ribbons 172 and set of short ribbons 174 can improve the operational integrity of the flexible circuit 100 by reducing or minimizing the chance of the plurality of traces 112 breaking at the proximal end 170.

The proximal end 170 can also have the two panel shield section 158. The two panel shield section 158 can extend to or extend beyond the set of long ribbons 172. The proximal end 170 can include a plurality of cut tabs 182. The plurality of cut tabs 182 can extend between the plurality of signal ribbons 130. The plurality of cut tabs 182 can keep the plurality of signal ribbons 130 aligned and in a fixed orientation in the unfolded configuration in order to prevent damage to the plurality of signal ribbons 130 or the plurality of traces 112. To fold the flexible circuit 100 the plurality of cut tabs 182 can be cut so that the plurality of signal ribbons 130 can be folded (e.g., rolled) in order to form the device for insertion into the catheter.

The proximal end 170 can also include fishing holes 178. The fishing holes 178 can be used to position the flexible circuit 100 in the catheter. For example, when in a folded configuration, the flexible circuit 100 can be coupled to or attached to a line or alignment device at the fishing holes 178. The line or alignment device can pull the flexible circuit 100 (e.g., by the fishing holes 178) through a shaft of a catheter during assembly. Additionally, the two panel shield section 158 at the proximal end 170 is soldered to enclose the proximal end 170 once the flexible circuit 100 is installed into the catheter.

FIGS. 7A-7C illustrate the flexible circuit 100 in the wrapped (e.g., rolled) configuration. The flexible circuit 100 can be wrapped by folding each section of the plurality of signal ribbons 130 on top of one another, followed by the shield regions (e.g., two panel shield section 158 and three panel shield section 160). For example, the first signal ribbon 130A can fold underneath the second signal ribbon 130B, then both the first signal ribbon 130A and the second signal ribbon 130B can fold underneath the third signal ribbon 130C, then, the first signal ribbon 130A, the second signal ribbon 130B, and the third signal ribbon 130C can fold underneath the fourth signal ribbon 130D in order to stack the plurality of signal ribbons 130. Additionally, the two panel shield section 158 and three panel shield section 160 can wrap around the stacked plurality of signal ribbons 130 in order to place the flexible circuit 100 in an assembled state (e.g., wrapped). The two panel shield section 158 and three panel shield section 160 can be external to the plurality of signal ribbons 130 in the wrapped configuration. The flexible circuit 100 can be inserted into the catheter in the wrapped configuration. The flexible circuit 100 is wrapped in two sections (e.g., the two panel shield section 158 and the three panel shield section 160). In the two panel shield section 158 adjacent to the crosshatch 134, there is no ground (e.g., copper) on the bottom later. In the three panel shield section 160, there is a ground (e.g., copper) pour on the bottom layer. The ground on the bottom layer in the wrapped configuration allows the plurality of signal ribbons 130 to be fully enclosed with some overlapping regions to account for bulging during folding. Additionally, the proximal end 170 can be tucked into the shield panels when the flexible circuit 100 is in a folded state.

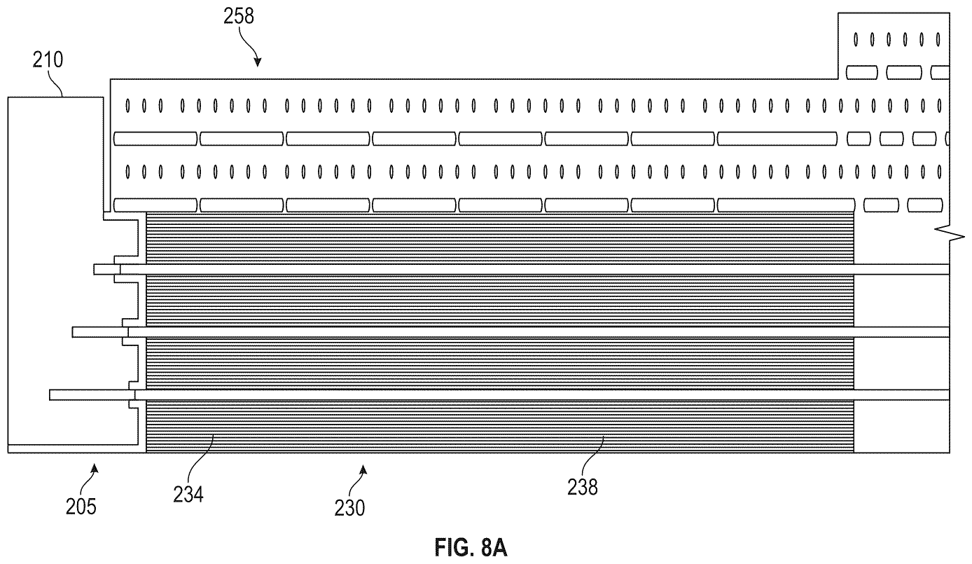

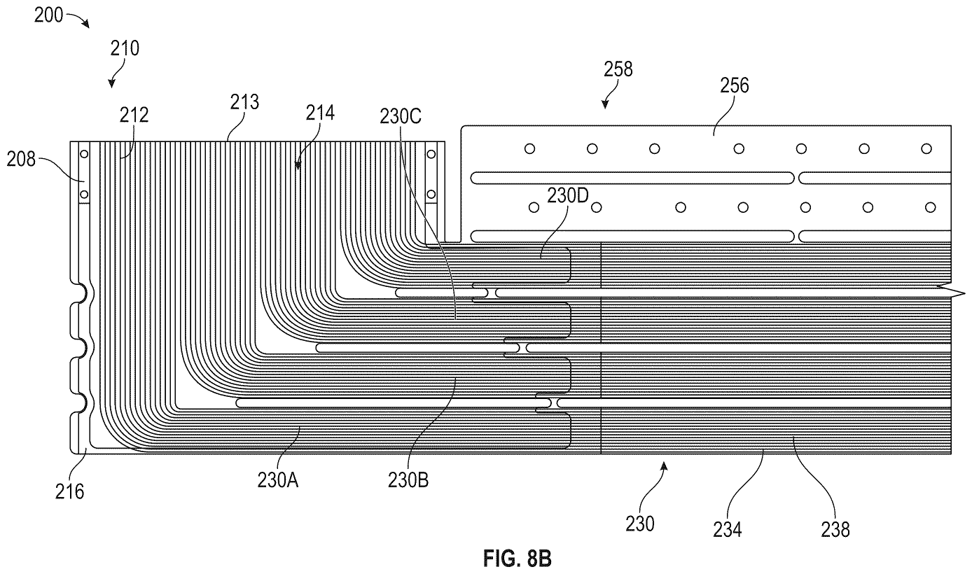

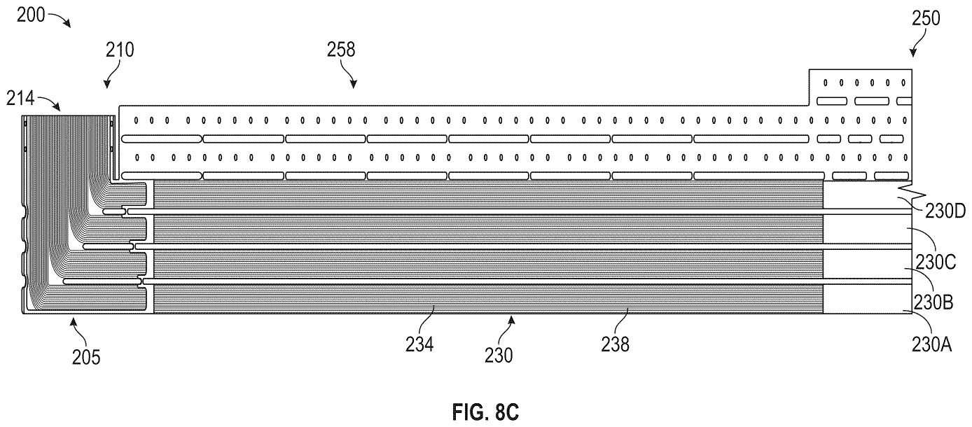

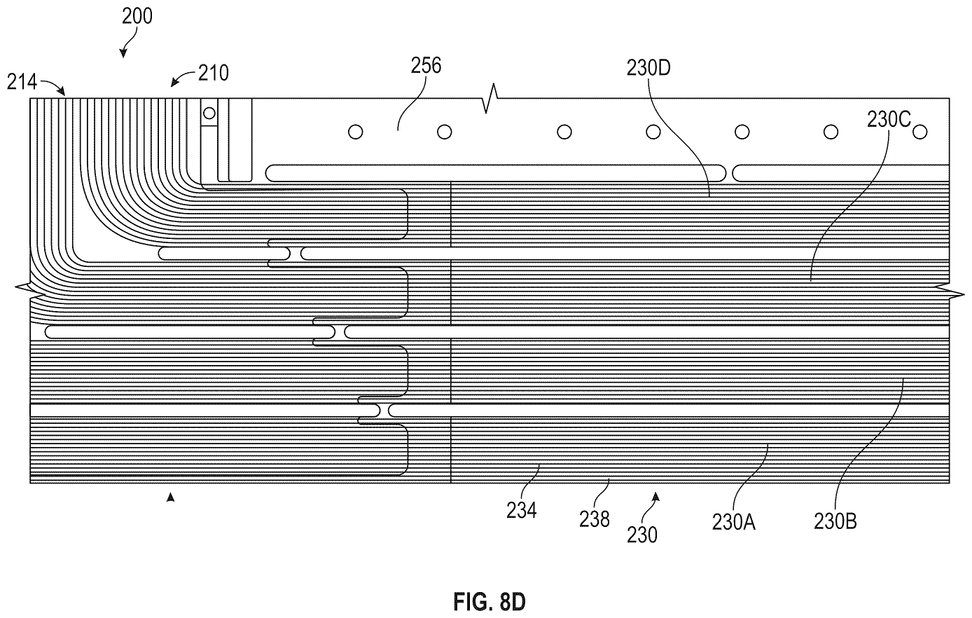

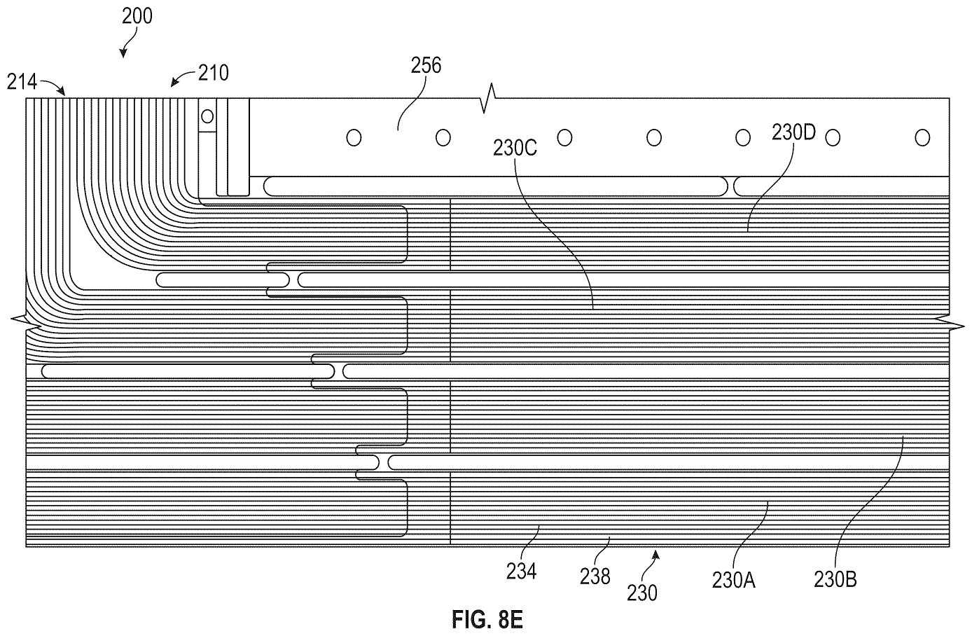

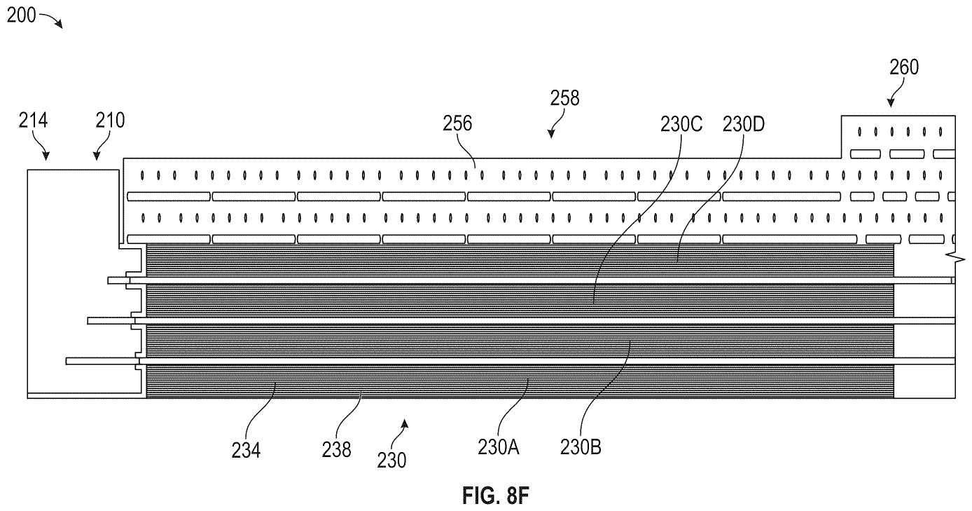

FIGS. 8A-8F illustrate another embodiment of a flexible circuit 200. In many aspects, the flexible circuit 200 is similar to the flexible circuit 100 described above and the same or similar reference numbers are used to refer to the same or similar features. For example, the flexible circuit 200 includes the distal end 210, the middle region 250, the two panel shield section 258, and the three panel shield section 260 which are similar to corresponding features described above. Additionally, the flexible circuit 200 includes a plurality of traces 212, a plurality of signal ribbons 230, and a ground bar 208 that are all similar or identical to the corresponding features described above.

FIGS. 8A-8F illustrate the distal end 210 of the flexible circuit 200. The distal end 210 can include a flexible region. The distal end 210 can have a transducer attachment 214. The length of the transducer attachment 214 extending from the distal end 210 to the beginning portion (e.g., the beginning portion being adjacent to the distal most edge of the two panel shield 256) of the two panel shield section 258 can have a length of approximately 12.3 millimeters. The length of the transducer attachment 214 extending from the distal end 210 to the beginning portion of the two panel shield section 258 can have a length of approximately 11 millimeters, 11.5 millimeters, 12 millimeters, 12.5 millimeters, 13 millimeters, 13.5 millimeters, or 14 millimeters. The transducer attachment 214 can include a plurality of traces 212. The plurality of traces 212 can extend from a top or upper region 213 of the transducer attachment 214 to the plurality of signal ribbons 230 (see FIG. 8B). Each trace of the plurality of traces 212 can be terminated by a 32-pin connector. Additionally, in some embodiments, half or approximately half of the plurality of traces 212 are electrically coupled to a ground portion or the ground bar 208. The transducer attachment 214 can be positioned adjacent to (e.g., horizontally of) the plurality of signal ribbons 230. The plurality of signal ribbons 230 can extend from the transducer attachment 214 at a position near or adjacent to the beginning portion (e.g. the beginning portion being adjacent to the distal most edge of the two panel shield 256. The plurality of signal ribbons 230 can be four signal ribbons, which can include a first signal ribbon 230A, a second signal ribbon 230B, a third signal ribbon 230C, and a fourth signal ribbon 230D. The transducer attachment 214 can have a ground section or ground bar 208 which can be positioned along an outside edge 216 of the transducer attachment 214. The top or upper region 213 can be approximately twelve millimeters. Additionally, the top or upper region 213 can be approximately nine millimeters, ten millimeters, eleven millimeters, thirteen millimeters, or fourteen millimeters. The outside edge 216 at a distalmost region can be approximately ten millimeters. Additionally, the outside edge 216 can be approximately eight millimeters, nine millimeters, eleven millimeters, twelve millimeters, or thirteen millimeters.

The plurality of signal ribbons 230 extending from the transducer attachment 214 on the distal end 210 can have horizontal bars 234. The horizontal bars 234 can improve the flexibility of the plurality of signal ribbons 230 at the distal end 210. The horizontal bars 234 can connect ground regions between the plurality of signal ribbons 230 when the flexible circuit 200 is in the folded configuration. The horizontal bars 234 can be positioned on the top cover layer 205. Additionally, the horizontal bars 234 can be operably connected to (e.g., physically or electrically coupled) the ground bar 208. The horizontal bars 234 can have a stripe-like pattern which can advantageously balance the amount of ground material along the plurality of signal ribbons 230 with a desired flexibility of the plurality of signal ribbons 230. The horizontal bars 234 can terminate at (e.g. end) at the middle region 250. The horizontal bars 234 can have a plurality of spaces (or “gaps”) 238 positioned along the plurality of signal ribbons 230 at the distal end 210 or flex region (e.g., the portion of the plurality of signal ribbons 230 adjacent to the transducer attachment 214). The spaces 238 are regions between the horizontal bars 234. The plurality of spaces 238 can be approximately 0.5 millimeters in length or width. In some embodiments, the plurality of spaces 238 can be approximately 0.35 millimeters, 0.4 millimeters, 0.45 millimeters, 0.55 millimeters, 0.6 millimeters in length or width. The plurality of spaces 238 can provide additional flexibility for the plurality of signal ribbons 230.

The foregoing description details certain embodiments of the systems, devices, and methods disclosed herein. It will be appreciated, however, that no matter how detailed the foregoing appears in text, the systems, devices, and methods can be practiced in many ways. As is also stated above, it should be noted that the use of particular terminology when describing certain features or aspects of the invention should not be taken to imply that the terminology is being re-defined herein to be restricted to including any specific characteristics of the features or aspects of the technology with which that terminology is associated.

It will be appreciated by those skilled in the art that various modifications and changes may be made without departing from the scope of the described technology. Such modifications and changes are intended to fall within the scope of the embodiments. It will also be appreciated by those of skill in the art that parts included in one embodiment are interchangeable with other embodiments; one or more parts from a depicted embodiment can be included with other depicted embodiments in any combination. For example, any of the various components described herein and/or depicted in the figures may be combined, interchanged or excluded from other embodiments.

As is also stated above, it should be noted that the use of particular terminology when describing certain features or aspects should not be taken to imply that the terminology is being re-defined herein to be restricted to including any specific characteristics of the features or aspects of the technology with which that terminology is associated. Conditional language such as, among others, “can,” “could,” “might” or “may,” unless specifically stated otherwise, are otherwise understood within the context as used in general to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Headings are included herein for reference and to aid in locating various sections. These headings are not intended to limit the scope of the concepts described with respect thereto. Such concepts may have applicability throughout the entire specification.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise.

For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, may represent endpoints or starting points, and ranges for any combination of the data points. For example, if a particular data point “10” and a particular data point “15” may be disclosed, it is understood that greater than, greater than or equal to, less than, less than or equal to, and equal to 10 and 15 may be considered disclosed as well as between 10 and 15. It is also understood that each unit between two particular units may be also disclosed. For example, if 10 and 15 may be disclosed, then 11, 12, 13, and 14 may be also disclosed.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

The phrase “based on” does not mean “based only on,” unless expressly specified otherwise. In other words, the phrase “based on” describes both “based only on” and “based at least on.” Unless otherwise explicitly stated, articles such as “a” or “an” should generally be interpreted to include one or more described items. Accordingly, phrases such as “a device configured to” are intended to include one or more recited devices.

It will be appreciated by those skilled in the art that various modifications and changes may be made without departing from the scope of the described technology. Such modifications and changes are intended to fall within the scope of the embodiments. It will also be appreciated by those of skill in the art that parts included in one embodiment are interchangeable with other embodiments; one or more parts from a depicted embodiment can be included with other depicted embodiments in any combination. For example, any of the various components described herein and/or depicted in the figures may be combined, interchanged or excluded from other embodiments.

The above description also discloses methods and materials of the present application. The catheter device described herein may be susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention as embodied in the attached claims. Applicant reserves the right to submit claims directed to combinations and sub-combinations of the disclosed inventions that are believed to be novel and non-obvious. Inventions embodied in other combinations and sub-combinations of features, functions, elements and/or properties may be claimed through amendment of those claims or presentation of new claims in the present application or in a related application. Such amended or new claims, whether they are directed to the same invention or a different invention and whether they are different, broader, narrower or equal in scope to the original claims, are to be considered within the subject matter of the inventions described herein.

What is claimed is:

1. A flexible circuit for insertion into a catheter device, comprising:
a distal region, comprising:
a transducer attachment comprising a plurality of traces for coupling to one or more transducers;
a plurality of signal ribbons extending from the transducer attachment, each of the plurality of signal ribbons comprising a set of the plurality of traces electrically coupled to and extending along a length of the plurality of signal ribbons; and
a first shielding section positioned above the plurality of signal ribbons in an unfolded configuration;
a medial region, comprising:
a second shielding section adjacent to the first shielding section; and
a plurality of vias positioned along the second shielding section;
a proximal region, comprising:
a set of long signal ribbons;
a second set of short signal ribbons adjacent to the set of long signal ribbons; and
a plurality of connector pads positioned on the set of long signal ribbons and the second set of short signal ribbons;
wherein the flexible circuit comprises a plurality of layers, wherein a first layer of the plurality of layers comprises a crosshatching section, the crosshatching section configured to connect ground regions between the plurality of signal ribbons; and
wherein the plurality of vias are configured to connect the ground regions across the plurality of layers, wherein the plurality of vias are configured the ground regions between at least a set of the plurality of signal ribbons.

2. The flexible circuit of claim 1, wherein the plurality of traces is sixty-four traces.

3. The flexible circuit of claim 1, wherein each set of the plurality of traces is sixteen traces.

4. The flexible circuit of claim 1, wherein the transducer attachment comprises a ground bar, wherein at least a portion of the plurality of traces are coupled to the ground bar.

5. The flexible circuit of claim 1, wherein the plurality of signal ribbons comprises four signal ribbons, wherein each of the four signal ribbons is electrically coupled to a set of the plurality of traces, each set of the plurality of traces comprising sixteen traces.

6. The flexible circuit of claim 1, wherein the crosshatching section extends from a distal end of the plurality of signal ribbons.

7. The flexible circuit of claim 1, wherein the plurality of layers includes five layers, wherein the first layer is a top ground layer, wherein a second layer is a top cover layer, wherein a third layer is a base polyimide layer, wherein a fourth layer is a bottom ground layer, wherein a fifth layer is a bottom cover layer.

8. The flexible circuit of claim 1, wherein the medial region is longer than the distal region and the proximal region.

9. The flexible circuit of claim 1, wherein the second shielding section includes three panels, wherein the second shielding section includes a plurality of slots configured to permit the flexible circuit to bend.

10. The flexible circuit of claim 1, wherein the plurality of signal ribbons are configured to roll into a folded configuration for assembly in the catheter device.

11. The flexible circuit of claim 1, wherein the plurality of vias include first vias and second vias, wherein the first vias form a cluster of three vias, wherein the second vias form a cluster of five vias.

12. The flexible circuit of claim 11, wherein the first vias and the second vias are operably positioned along the second shielding section to reduce a distance between ground regions when the flexible circuit is in a folded configuration.

13. The flexible circuit of claim 1, wherein the plurality of traces extend from the transducer attachment, along an entire length of the plurality of signal ribbons, and terminates at the plurality of connector pads.

14. The flexible circuit of claim 1, wherein the plurality of connector pads include a third set of vias, wherein the plurality of traces extending from the transducer attachment terminate the third set of vias.

15. The flexible circuit of claim 1, wherein the first shielding section and the second shielding section are configured to wrap around the plurality of signal ribbons.

16. The flexible circuit of claim 1, wherein a fishing hole extends through the plurality of signal ribbons in the proximal region and is configured to guide the flexible circuit inside the catheter device during assembly.

17. A flexible circuit for insertion into a catheter device, comprising:
a distal region, comprising:
a transducer attachment comprising a plurality of traces;
a plurality of signal ribbons extending from the transducer attachment, each of the plurality of signal ribbons comprising a set of the plurality of traces electrically coupled to and extending along a length of the plurality of signal ribbons; and
a first shielding section positioned above the plurality of signal ribbons in an unfolded configuration;
a medial region, comprising:
a second shielding section adjacent to the first shielding section; and
a plurality of vias positioned along the second shielding section;
a proximal region comprising a plurality of connector pads positioned on the plurality of signal ribbons;
wherein the flexible circuit comprises a plurality of layers, wherein a first layer of the plurality of layers comprises a crosshatching section, the crosshatching section configured to connect ground regions between the plurality of signal ribbons; and
wherein the plurality of vias are configured to connect the ground regions between at least a set of the plurality of signal ribbons.

18. The flexible circuit of claim 17, wherein the plurality of signal ribbons are configured to roll into a folded configuration for assembly in the catheter device.

19. The flexible circuit of claim 17, wherein the plurality of vias include first vias and second vias, wherein the first vias form a cluster of three vias, wherein the second vias form a cluster of five vias.

20. The flexible circuit of claim 17, wherein the plurality of connector pads include a third set of vias, wherein the plurality of traces extending from the transducer attachment terminate the third set of vias.
